(19) United States
(12) Patent Application Publication
(10) Pub. No.: US 2021/0335468 A1
(43) Pub. Date: Oct. 28, 2021

(54) ELECTRONIC SYSTEM FOR AUTOMATICALLY RECOMMENDATING PHARMACY STORES ALL SUITABLE DRUG PRODUCTS AND METHODS THEREOF

(71) Applicant: EcoScript LLC, St. Augustine, FL (US)

(72) Inventor: Roy H. Hinman II, Palm Coast, FL (US)

(21) Appl. No.: 17/241,995

(22) Filed: Apr. 27, 2021

Related U.S. Application Data
(60) Provisional application No. 63/016,047, filed on Apr. 27, 2020.

(51) Int. Cl. G16H 20/10; G16H 50/20; G16H 80/00

(52) U.S. Cl. CPC G16H 20/10; G16H 80/00; G16H 50/20

(57) ABSTRACT

The present invention discloses an electronic system for providing online clinic information to patients and employees of clinics. Especially the patient and the doctor can select suitable drug products and pharmacies among prioritized lists which are automatically provided by the electronic system according to a recommendation policy. The recommendation policy is predetermined according to several considerations such as availabilities and costs of the suitable drug products for identical purpose of medical treatment.

### CROSS REFERENCE TO RELATED PATENT APPLICATION

This patent application is based on a provisional patent application No. 63/016,047 filed on Apr. 27, 2020.

### FIELD OF THE INVENTION

The present invention relates to electronic system, and more particularly, to electronic system for providing online clinic information to patients and employees of clinics and recommending pharmacy stores all suitable drug products.

### BACKGROUND OF THE INVENTION

Due to tightly controlled privacy and other professional issues, the patient's record is usually not accessible to patients themselves, and so is to the medical professional employees such as front desk clerks, technicians, nurses, and doctors. Charts and papers are usually provided in paper forms. Daily operations of a clinic or a hospital rely on different separate systems. It requires large amount of works to integrate the outputs or records of these separate systems.

Hence there exists a need of an electronic system for providing online clinic information to patients and medical professional employees. Especially the patient and the doctor can select suitable drug products and pharmacies among prioritized lists which are automatically provided by the electronic system according to a recommendation policy.

### SUMMARY OF THE INVENTION

An objective of the present invention is to provide an electronic system for providing online clinic information to patients and employees of clinics. Especially the patient and the doctor can select suitable drug products and pharmacies among prioritized lists which are automatically provided by the electronic system according to a recommendation policy. The recommendation policy is predetermined according to several considerations such as availabilities and costs of the suitable drug products for identical purpose of medical treatment.

A system is provided according to the present invention. The system comprising a first computer and a second computer. The first computer is configured for receiving one or more purposes for medical treatment from a first user of the first computer and transmitting the one or more purpose to a second computer; providing prioritized suitable drug products for each one of the received purposes to the first user, wherein the suitable drug products are transmitted from the second computer; receiving the first user's command for selecting suitable drug products for each one of the received purposes; and sending the selected suitable drug products to the second computer. The second computer is configured for determining the prioritized suitable drug products for each one of the received purposes received from the first computer according to a recommendation policy; transmitting the prioritized suitable drug products for each one of the received purposes to the first computer; receiving the selected suitable drug products from the first computer; and producing an e-prescription according to the selected suitable drug products.

A method is provided according to an aspect of the present invention. The method comprising: receiving one or more purposes for medical treatment from a first user of a first computer and transmitting the one or more purposes to a second computer; providing prioritized suitable drug products for each one of the received purposes to the first user, wherein the suitable drug products are transmitted from the second computer; receiving the first user's command for selecting suitable drug products for each one of the received purposes; and sending the selected suitable drug products to the second computer.

A method is provided according to an aspect of the present invention. The method comprising: determining prioritized suitable drug products for each one of purposes received from a first computer according to a recommendation policy; transmitting the prioritized suitable drug products for each one of the received purposes to the first computer; and receiving the selected suitable drug products from the first computer; and producing an e-prescription according to the selected suitable drug products.

A system is provided according to an aspect of the present invention. The system comprising a first computer and a second computer. The first computer is configured for: receiving one or more purposes for medical treatment from a first user of the first computer and transmitting the one or more purpose to a second computer; providing prioritized pharmacies store suitable drug products for each one of the received purposes to the first user, wherein the prioritized pharmacies stores suitable drug products for each one of the received purposes are transmitted from the second computer; receiving the first user's command for selecting one of the prioritized pharmacies and corresponding suitable drug products for each one of the received purposes; and sending the selected pharmacy and corresponding suitable drug products for each one of the received purposes to the second computer. The second computer is configured for: determining the suitable drug products for each one of the received purposes received from the first computer according to a recommendation policy; determining the prioritized pharmacies store the suitable drug products for each one of the received purposes according to the recommendation policy; transmitting the prioritized pharmacies store the suitable drug products for each one of the received purposes to the first computer; receiving the selected pharmacy and corresponding suitable drug products for each one of the received purposes from the first computer; and producing an e-prescription according to the selected pharmacy and corresponding suitable drug products for each one of the received purposes.

A method is provided according to an aspect of the present invention. The method comprising: receiving one or more purposes for medical treatment from a first user of a first computer and transmitting the one or more purpose to a second computer; providing prioritized pharmacies store suitable drug products for each one of the received purposes to the first user, wherein the prioritized pharmacies stores suitable drug products for each one of the received purposes are transmitted from the second computer; receiving the first user's command for selecting one of the prioritized pharmacies and corresponding suitable drug products for each one of the received purposes; and sending the selected pharmacy and corresponding suitable drug products for each one of the received purposes to the second computer.

A method is provided according to an aspect of the present invention. The method comprising: determining suitable drug products for each one of purposes received from a first computer according to a recommendation policy; determining prioritized pharmacies store the suitable drug products for each one of the purposes according to the recommendation policy; transmitting the prioritized pharmacies store the suitable drug products for each one of the purposes to the first computer; receiving the selected pharmacy and corresponding suitable drug products for each one of the received purposes from the first computer; and producing an e-prescription according to the selected pharmacy and corresponding suitable drug products for each one of the purposes.

### BRIEF DESCRIPTION OF THE DRAWINGS

The advantages and spirit related to the present invention can be further understood via the following detailed description and drawings.

FIG. 1 shows an electronic system 100 for online clinic information to patients and employees of clinics in accordance with an embodiment of the present invention.

FIG. 2 shows a diagram including two tables and a view of a PDB (pharmaceutical database) 200 in accordance with an embodiment of the present invention.

FIG. 3 depicts a PDB 300 including purposes, drugs, pharmacies and available drugs of particular pharmacy tables in accordance with an embodiment of the present invention.

FIG. 4 shows a flowchart diagram of a method for recommending drug products in accordance with the present invention.

FIG. 5 shows a flowchart diagram of a method for recommending pharmacy and corresponding drug products in accordance with the present invention.

### DETAILED DESCRIPTION OF THE PREFERRED EMBODIMENT

Some embodiments of the present application are described in details below. However, in addition to the description given below, the present invention can be applicable to other embodiments, and the scope of the present invention is not limited by such rather by the scope of the claims. Moreover, for better understanding and clarity of the description, some components in the drawings may not necessary be drawn to scale, in which some may be exaggerated related to others, and irrelevant. If no relation of two steps is described, their execution order is not bound by the sequence as shown in the flowchart diagram.

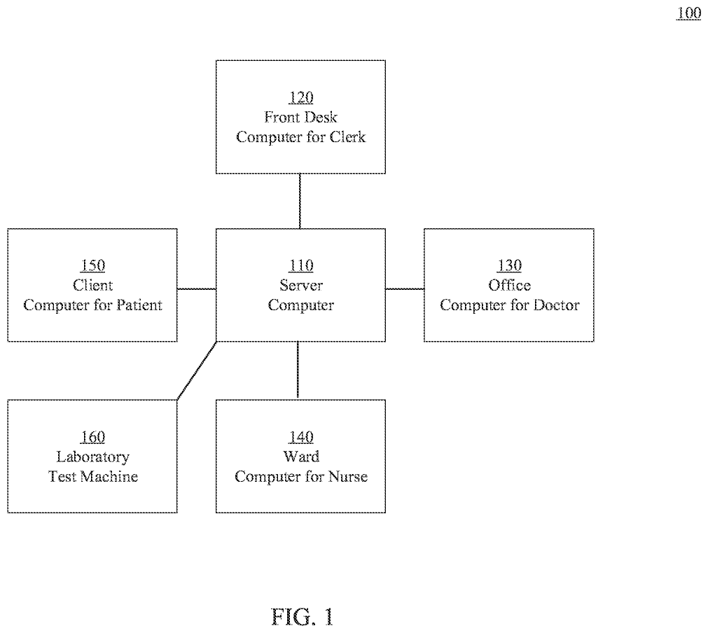

Please refer to FIG. 1, which shows an electronic system 100 for online clinic information to patients and employees of clinics in accordance with an embodiment of the present invention. The electronic system 100 may include a server computer 110, a front desk computer 120 for clerk, an office computer 130 for doctor, a ward computer 140 for nurse, a client computer 150 for patient and a laboratory test machine 160.

The server computer 110 may comprise one or more interconnected computing apparatuses. Each of the interconnected computing apparatuses may serve one or more modular computing functions such as implementing database management system (DBMS), business intelligence (BI), application interfacing functions (client/server architecture or web/browser architecture). The interconnected computing apparatuses may be implemented in psychical high performance server computers, virtual servers in a remote server farm or in a cloud services. In order to achieve high reliability, these interconnected computing apparatuses may implement redundancy or highly fault tolerance functionalities in order safeguard important medical and private data. Communications between the interconnections of these computing apparatuses may be encrypted in order to prevent invasions or hackings. Communication protocols provided by the modular functions may be compliant to industrial standard communication protocols, such as HTTP, HTTPS, ODBC, JDBC and etc. or may be proprietary protocols unknown to other systems.

In an embodiment following web/browser architecture, the server computer 110 provides HTTP and HTTPS interfaces to the front desk computer 120 for clerk, the office computer 130 for doctor, the ward computer 140 for nurse, the client computer 150 for patient. Those computers 120 through 150 mainly use web browsers to communicate with the server computer 110 using HTML over HTTP/HTTPS. In one embodiment, the server computer 110 may communicate with the laboratory test machine 160 by any protocols which may include HTML over HTTP/HTTPS.

For a web-browser architecture, the computers 120 through 150 and the laboratory test machine 160 may connect to peripheral devices such as input and/or output devices including but not limiting to geographical locator, GPS receiver, scanners, cameras, card readers, fingerprint/eye/facial scanners, speakers and any biological feature measurement instruments. These peripheral devices may be connected to the browsers run on the computers 120 through 150 by plug-in software for the browsers so as the data gathered from the input peripheral devices can be sent to the server computer 110 via the plug-in software of the browsers. Alternatively, for dedicated client/server architecture, installed special tailored applications for the computers 120 through 150 may connect to the input peripheral device via driver software installed in operating systems of the computers 120 through 150. Hence, the data gathered from the input peripheral devices can be sent to the server computer 110 via the driver software and the special tailored applications.

The computers 120 through 150 may be desktop, laptop, and mobile computer, pad, smartphone, personal digital assistant, or any other computing devices which can be connected to the server computer 110 via communication wires, personal area network, wireless local area networks, telecommunication networks or any other psychical links. The computers 120 through 150 may include input/output devices including but not limiting to display, touch screen, audio speaker, microphone

In a preferred embodiment, the client computer 150 is a mobile computer, a pad or a smartphone. A dedicated application may be installed in the client computer 150 for patient's function. Alternatively, a browser may be installed in the client computer 150 for connecting with the server computer 110. A patient may use the client computer 150 for requesting services of the electronic system 100. The services provided by the electronic system 100 or the server computer 110 may include following functions: view his/her entire history, laboratory reports, e-prescription, MAR (medical administrative record), EHR (electronic health record), the pharmacy based on location where the client computer resides, personal/family insurance/consent details, ancillaries provided till date, telemedicine options according to available video conference software and hardware provided by the client computer 150 and etc. In addition, the patient may view, modify, and cancel appointments in the electronic system 100.

The above mentioned records or data provided by the electronic system 100 and the server computer 110 are corresponding to a unique key so-called MRN (medical record number) assigned to one and only one patient. In order to provide privacy and security, the communication link between the client computer 150 and the server computer 110 may be encrypted, for example, encrypted by any industrial standard protocols such as HTTPS and TLS (transport layer security protocols). Besides, in order to enhance identification security of patient, the client computer 150 may include biological feature measurement instruments such as fingerprint/eye/facial scanners or voice recognizer to identify and to recognize the patient. Once the feature gathered by the instruments of the client computer 150 is matched to the recorded one corresponding to the associated MRN, the patient is allowed to access the above mentioned functions. Alternatively, the client computer 150 may include a card reader to read a security card of a patient. In case the security card is successfully read and decrypted, the patient is able to access the above mentioned functions provided by the electronic system 100 or the server computer 110. One or more types of security measures may be taken for increasing accountability and reliability of the electronic system 100.

The client computer 150 may be used to track where the patient is inside the hospital and the clinic. For example, wireless base stations installed in the hospital or the clinic may be used to provide wireless access to the client computer 150. Therefore the position of the wireless base station providing access function to the client computer 150 may be used to positioning the patient.

Furthermore, the client computer 150 may be located outside the hospital or the clinic operating the server computer 110. In addition, a virtual private network (VPN) may be used for the client computer 150 to the server computer 110. Once the patient carrying the client computer 110 enters perimeters of the hospital or the clinic, a secure wireless LAN may be used for interconnecting the server computer 110 and the client computer 150. The computers 110 and 140 may be resides in a private and secure network operated by the hospital and the clinic. However, although the network environment seems secure, the above mentioned card readers or biological feature measurement instruments may be also used in the computers 110 through 140 and the laboratory test machine 160 for granting access of employees including doctors, nurses, clerks and technicians.

In a preferred embodiment, the data stored in the DBMS of the server computer 110 may be encrypted. Just immediately before they are retrieved, decryption procedure is applied to the retrieved data to generate data shown in the computers in the electronic system 100. Any suitable data encryption/decryption algorithms and hardware may be used in the electronic system 100.

In a preferred embodiment, the patient's charts or medical records stored in the DBMS are organized or conformed to SOAP (subjective, objective, assessment and plan) notes and/or POMR (problem-oriented medical record). SOAP notes may include subjective component, objective component, assessment component and plan component. In the patient's database, MAR or HER may include following information: current issues of patient, patient personal detailed information, patient history, appointments, ancillaries, supplies, laboratory reports, care plans, charts, and e-prescriptions. Insurance and billing information may be also kept in the patient's database.

Currently, the data is managed through paper forms that are saved and collected in physical file folders. It is a time consuming job to find a patient's data in the particular file folder. And it is difficult to share the patient's data. i.e., the file folder, among the employees including doctors, nurses, clerks and technicians attending the patient concurrently. In one embodiment of the present invention, the data can be stored and managed in a cloud environment or in a DBMS provided by the server computer 110. It is easily to access the patient's data in few finger strokes on keyboards of the computers 110 through 140. It also allows concurrently views of the same data of identical patient. Once the data is updated by authorized employee, the updated data can be immediately propagated to other computers. Moreover, it is easier to train a new employee how to access digital online information system rather than finding and reading file folders with many informalities and versions.

A clerk may log in the front desk computer 120 for following functions: check schedule of a patient or a doctor for a particular date, view patient database, view and action on MAR of any patient's record, view comments in Nurses' Notes, view care plan of a patient, view laboratory reports of a patient and etc. The clerk may assist and modify patient with appointment and ancillary information.

A nurse may log in the ward computer 140 for almost every functions corresponding to a clerk. Additionally, a nurse is further authorized to write into Nurses' Notes, care plan of a patient, and assist doctor or patient with any process or procedure.

A doctor may log in the office computer 130 for following functions: schedule his/her own daily check, telemedicine by the office computer 130, access the patient information like history and current issues, access Nurses' Notes, access MAR of any patient's record, generate e-prescription, view and validate laboratory report, recommend patient to laboratory, refer patient to other doctors which have accounts in the electronic system 100 and etc. In one embodiment, a handwriting function may be provided by the office computer 130 for recognizing prescription and/or chart written by the doctor. The prescription would be transformed into e-prescription with the help of word recognizer. In a preferred embodiment, the recognizer is enhance by AI (artificial intelligence) implemented by software and/or special tailored hardware.

When producing e-prescription, the electronic system 100 or the server computer 110 may provide drug information and drug/salt recommendation to the doctor. In addition, the electronic system 100 may provide available pharmacies nearby the patient's location and suggestions. It may recommend patients to in-house pharmacies based on criticality and/or symptoms. Furthermore, alternative drugs, continue drugs and discontinue drugs may be provided by the electronic system 100 or the server computer 110. Later in the present invention, a detailed discussion of the pharmaceutical issues would be addressed.

A technician or an operator for operating the laboratory test machine 160 may also log in for providing test reports to the electronic system 100 and server computer 110. Once the laboratory test reports generated by the laboratory test machine 160, the test report and the technician/operator may be included in the patient's data after validation of the doctor. The laboratory test machine 160 may be one of following instruments: X-ray photo machine, CT (computerized tomography) machine, MRI (magnetic resonance imaging) machine, PET (positron emission tomography), electrocardiography monitor and etc.

In addition to clerks, doctors, nurses, technicians and patients, an administrator of the electronic system 100 may have following functions: access data of the employees including clerks, doctors, nurses, technicians and patients, view entire history of patients, laboratory reports, e-prescriptions, MARs, EHRs, personal/family insurance consent details and the ancillary provided till date. Furthermore, the administrator may be able to modify appointments and manage drug and pharmacy information.

In order to provide recommendation of drug and pharmacy for a patient to a doctor, the electronic system 100 may include a PDB (pharmaceutical database) for matching brand named products with generic products used for the same purpose. The PDB may also record the market prices of these brand-named products and generic products for the same purposes. Various side effects and any other additional notes and comments for the drugs corresponding to the same purpose are also maintained in the PDB.

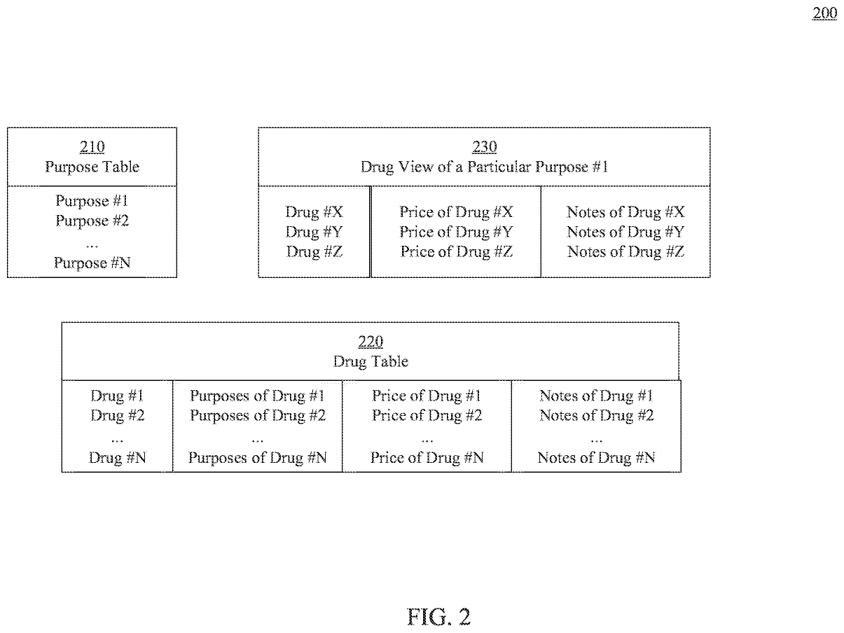

Please refer to FIG. 2, which shows a diagram including two tables and a view of a PDB (pharmaceutical database) 200 in accordance with an embodiment of the present invention. There are two tables included in the PDB for recording drug information and purpose of drug information. A purpose table 210 includes one or more medical purposes. For example, purposes like painkilling, anti-inflammatory anti-fever and etc. And a drug table 220 includes one or more brand-named products or generic products, its purposes, market prices and notes. Although the tables 210 and 220 shown in FIG. 2 merely include few properties of purposes and drugs, more fields or columns of the purpose table 210 and the drug table 220 may be added to the tables 210 and 220 to accommodate the requirements of real world applications. Besides, the data schema design in the PDB may be normalized in alternative formats. However, the present invention only requires reciting correspondences between multiple drugs with price tags and their common purposes. The tables 210 and 220 may be maintained by the administrator. In a preferred embodiment, the prices may be automatically imported from other external or internal information system.

As shown in FIG. 2, a view 230 dedicated to drugs for a particular purpose could be statically or dynamically generated according to the tables 210 and 220 of the PDB. For example, drugs #X, #Y and #Z share a common purpose #1. According to the view 230, the doctor may select the most expensive drug because the patient's insurance covers. Alternatively, the doctor may select drug #Z because the patent had bad experiences to drug #X and drug #Y. In one embodiment, the doctor may avoid drug #Y because it is conflicted with other drug serving other purpose.

In a preferred embodiment, a business intelligence module or a drug recommender selects the drugs based on a set of rules, which may include prices, side effects, patient's allergy history and any other considerations. The drug recommender may sort the results in several priority lists with different considerations. The doctor and/or the patient may select one drug for the purpose. The business intelligence module may use a kind of easy use plain text script languages to denote the rules. Doctors and/or administrators may write standard operating procedures (SOPs) for the drug selecting or sorting rules. In an embodiment, the rules or the script language may be generated in a user friendly graphical user interface including drag and drop function and supporting touch screen functions.

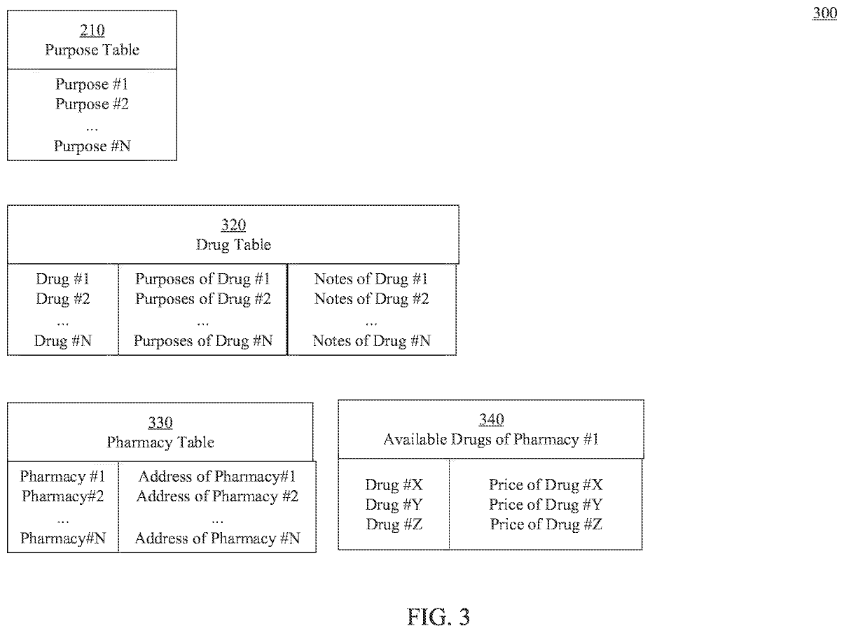

Please refer to FIG. 3, which depicts a PDB 300 including purposes, drugs, pharmacies and available drugs of particular pharmacy tables in accordance with an embodiment of the present invention. Comparing with the drug table 220 as shown in FIG. 2, the drug table 320 as shown in FIG. 2 is lack of the price field. This is because the price tags of each drug are recorded in the available drugs of particular table 340. And the prices of identical drug product available in different pharmacies may be different. A pharmacy table 330 is used to denote tuples of pharmacies and their addresses. More information of the pharmacies such as open hour and telephone may be denoted in more fields.

A business intelligence module or a recommender may select a most suitable pharmacy according to data recorded in the PDB 300 including one or more logics such as availabilities of drugs in the e-prescription, total costs of drugs sold by the pharmacy, minimum distance between the pharmacy and patient's current location or home address, and etc. Patient's current location or home may be gathered by a geo locating service provided by the client computer 150 or retrieval from patient's database. The recommender may also consider the prices of drugs, side effects, patient's allergy history and any other reasons which already mentioned in the embodiment as shown in FIG. 2.

The availabilities of drugs and the prices of each pharmacy recorded in the PDB 300 may be manually maintained by human administrators or imported from external systems. For example, the pharmacy may automatically report the above mentioned information via an interface. Or, in the contrary, the electronic system 100 may collect or query the information from the pharmacies periodically. There may exist an in-house pharmacy collocate with the hospital or the clinic. The in-house pharmacy may have the first priority.

The pharmacy and the electronic system 100 may exchange and validate the e-prescriptions. Valid e-prescription of the patient may be shown by the client computer 150. After reviewing the valid e-prescription shown on the client computer 150, the pharmacy may check whether this e-prescription is valid or not via the communication link of the electronic system 100. Once the drug products of the e-prescription are taken by the patient, the pharmacy may send the message to the electronic system 100 to expire or to invalidate the e-prescription. After the e-prescription is expired or invalidated, the electronic system 100 would response to another inquiry message from another pharmacy that the e-prescription is already invalidated. The second or later pharmacy should not let the patient to take or buy drug products. The client computer 150 may also indicate the e-prescription is already expired or invalidated.

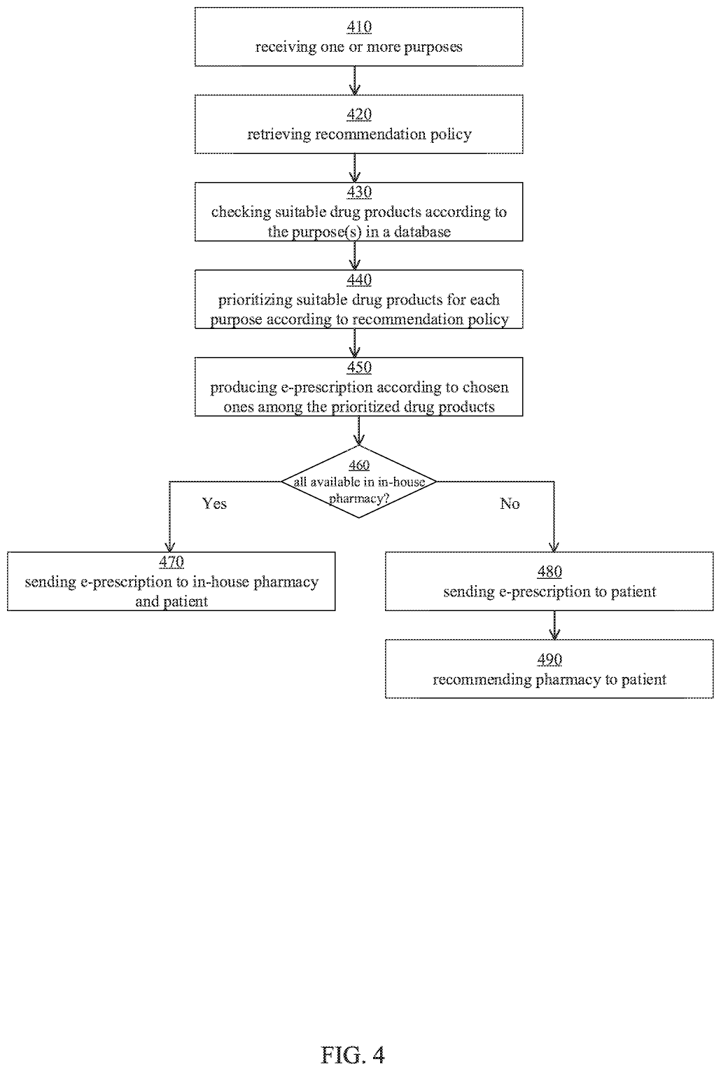

Please refer to FIG. 4, which shows a flowchart diagram of a method for recommending drug products in accordance with the present invention. The method may be implemented as instructions stored in non-volatile memory which are executable for at least one of the server computer 110 and any suitable computers in the electronic system 100. The method may include following steps. Unless two steps have causal relation, the present invention does not limit the order of these two steps.

Step 410: receiving one or more purposes. The purposes may be selected from the purpose table 210 as shown in FIGS. 2 and 3.

Step 420: retrieving recommendation policy. The recommendation policy used for selecting or prioritizing suitable drug products may be stored in the DBMS of the server computer 110 or may be dynamically selected by doctor. For example, the recommendation policy may be a lowest cost policy, a most profitable policy, a least aggressive policy or a best fit policy.

Step 430: checking suitable drug products for each of the received purposes in the database such as the PDB 200. The drug products may be selected from the drug table 220 as shown in FIG. 2.

Step 440: prioritizing suitable drug products for each of the received purposes according to the retrieved recommendation policy. In one example, the recommendation policy considers price/cost or side effects of the drug products.

Step 450: producing e-prescription according to chosen ones among the prioritized drug products.

Optional step 460: determining whether the drug products listed in the produced e-prescription available in an in-house pharmacy. If the result of this determination is positive, the flow goes to step 470. Otherwise, the flow goes to step 480.

Step 470: sending the produced e-prescription to an information system of the in-house pharmacy and the patient. Thus the patient can go to the in-house pharmacy to take the drug products. In addition, a clerk and/or a nurse may assist the patient to take the drug products listed in the e-prescription by his/her own computer. Once the drug products of the e-prescription are taken, the in-house pharmacy may send message to expire the e-prescription in the electronic system 100.

Step 480: sending the produced prescription/e-prescription to patient. The flow may further go to optional step 490.

Optional step 490: recommending pharmacy to patient. The recommendation may be based on one or more considerations such as distance between the patient's current location or home address and the pharmacy, availabilities of drug products listed in the prescription, price of drug products in the pharmacy, and etc.

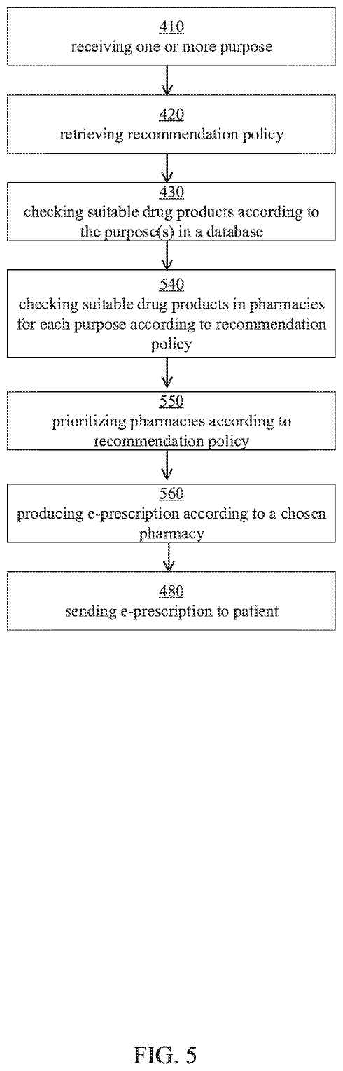

Please refer to FIG. 5, which shows a flowchart diagram of a method for recommending pharmacy and corresponding drug products in accordance with the present invention. The method may be implemented as instructions stored in non-volatile memory which are executable for at least one of the server computer 110 and any suitable computers in the electronic system 100. The method may include the above mentioned steps 410, 420, 430 and 480 and following steps. Unless two steps have causal relation, the present invention does not limit the order of these two steps.

Step 540: checking suitable drug products in pharmacies for each one of the received purposes according to the retrieved recommendation policy. The step 540 is similar to the step 440. However, the recommendation policy may further consider availabilities and/or the prices of the drug products in the pharmacies. In an example, in case a drug product is not available in many pharmacies, alternative drug product available in many pharmacies may be prioritized instead. In another example, in case a drug product is too expensive, alternative drug product available may be prioritized. There are many variants and examples of recommendation policies which cannot be enumerated and elaborated in the present invention.

Step 550: prioritizing pharmacies for the retrieved recommendation policy. Similar to the step 490, the recommendation policy may be based on one or more considerations such as distance between the patient's current location or home address and the pharmacy, availabilities of drug products listed in the prescription, price of drug products in the pharmacy, and etc.

Step 560: producing e-prescription according to a chosen one of the prioritized pharmacies. At last, the produced e-prescription/prescription is sent to the patient at step 480.

According to an embodiment of the present invention, providing a system comprising a first computer and a second computer. The first computer is configured for receiving one or more purposes for medical treatment from a first user of the first computer and transmitting the one or more purpose to a second computer; providing prioritized suitable drug products for each one of the received purposes to the first user, wherein the suitable drug products are transmitted from the second computer; receiving the first user's command for selecting suitable drug products for each one of the received purposes; and sending the selected suitable drug products to the second computer. The second computer is configured for determining the prioritized suitable drug products for each one of the received purposes received from the first computer according to a recommendation policy; transmitting the prioritized suitable drug products for each one of the received purposes to the first computer; receiving the selected suitable drug products from the first computer; and producing an e-prescription according to the selected suitable drug products.

Preferably, in order to reduce costs of the e-prescription, the recommendation policy considers prices of the suitable drug products for each one of the purposes.

Preferably, in order to check whether the in-house pharmacy has all of the drug products of the e-prescription, the second computer is further configured for: determining wherein the selected suitable drug products in the e-prescription are all available in a first pharmacy; and sending the e-prescription to an information system of the first pharmacy in case the selected suitable drug products in the e-prescription are determined all available in the first pharmacy.

Preferably, in order to let patient to have the e-prescription on his/her own client computer, the second computer is further configured for: storing the e-prescription in case the selected suitable drug products in the e-prescription are not all available in the first pharmacy; and sending the e-prescription to a third computer of the system configured for displaying the e-prescription.

Preferably, in order to let patient to go to just one pharmacy to take all the drug products in the e-prescription, the second computer is further configured for selecting and transmitting a second pharmacy among a plurality of pharmacies where store all of the selected suitable drug products in the e-prescription to the third computer or the first computer.

Preferably, in order to let patient to go to just one nearest pharmacy to take all the drug products in the e-prescription, the second pharmacy is selected among the plurality of pharmacies based on a minimum distance between a location of the third computer and each of the plurality of pharmacies.

According to an embodiment of the present invention, providing a method comprising: receiving one or more purposes for medical treatment from a first user of a first computer and transmitting the one or more purposes to a second computer; providing prioritized suitable drug products for each one of the received purposes to the first user, wherein the suitable drug products are transmitted from the second computer; receiving the first user's command for selecting suitable drug products for each one of the received purposes; and sending the selected suitable drug products to the second computer.

Preferably, in order to reduce costs of the e-prescription, the recommendation policy considers prices of the suitable drug products for each one of the purposes.

According to an embodiment of the present invention, providing a method comprising: determining prioritized suitable drug products for each one of purposes received from a first computer according to a recommendation policy; transmitting the prioritized suitable drug products for each one of the received purposes to the first computer; and receiving the selected suitable drug products from the first computer; and producing an e-prescription according to the selected suitable drug products.

Preferably, in order to reduce costs of the e-prescription, the recommendation policy considers prices of the suitable drug products for each one of the purposes.

Preferably, in order to check whether the in-house pharmacy has all of the drug products of the e-prescription, the method further comprises: determining wherein the selected suitable drug products in the e-prescription are all available in a first pharmacy; and sending the e-prescription to an information system of the first pharmacy in case the selected suitable drug products in the e-prescription are determined all available in the first pharmacy.

Preferably, in order to let patient to have the e-prescription on his/her own client computer, the method further comprises: storing the e-prescription in case the selected suitable drug products in the e-prescription are not all available in the first pharmacy; and sending the e-prescription to a third computer of the system configured for displaying the e-prescription.

Preferably, in order to let patient to go to just one pharmacy to take all the drug products in the e-prescription, the method further comprises: recommending and transmitting a second pharmacy among a plurality of pharmacies where store all of the selected suitable drug products in the e-prescription to the third computer or the first computer.

Preferably, in order to let patient to go to just one nearest pharmacy to take all the drug products in the e-prescription, the second pharmacy is selected among the plurality of pharmacies based on a minimum distance between a location of the third computer and each of the plurality of pharmacies.

According to an embodiment of the present invention, providing a system comprising a first computer and a second computer. The first computer is configured for: receiving one or more purposes for medical treatment from a first user of the first computer and transmitting the one or more purpose to a second computer; providing prioritized pharmacies store suitable drug products for each one of the received purposes to the first user, wherein the prioritized pharmacies stores suitable drug products for each one of the received purposes are transmitted from the second computer; receiving the first user's command for selecting one of the prioritized pharmacies and corresponding suitable drug products for each one of the received purposes; and sending the selected pharmacy and corresponding suitable drug products for each one of the received purposes to the second computer. The second computer is configured for: determining the suitable drug products for each one of the received purposes received from the first computer according to a recommendation policy; determining the prioritized pharmacies store the suitable drug products for each one of the received purposes according to the recommendation policy; transmitting the prioritized pharmacies store the suitable drug products for each one of the received purposes to the first computer; receiving the selected pharmacy and corresponding suitable drug products for each one of the received purposes from the first computer; and producing an e-prescription according to the selected pharmacy and corresponding suitable drug products for each one of the received purposes.

Preferably, in order to let patient to go to just one pharmacy to take all the drug products in the e-prescription and to lower cost, the recommendation policy considers availabilities and prices of the suitable drug products for each one of the purposes provided in the prioritized pharmacies.

Preferably, in order to let patient to go to just one nearest pharmacy to take all the drug products in the e-prescription, the recommendation policy further considers a minimum distance between the prioritized pharmacies and a location of a third computer of the system configured for displaying the e-prescription.

Preferably, in order to let patient to have the e-prescription on his/her own client computer, the second computer is further configured for sending the e-prescription to a third computer of the system configured for displaying the e-prescription.

According to an embodiment of the present invention, providing a method comprising: receiving one or more purposes for medical treatment from a first user of a first computer and transmitting the one or more purpose to a second computer; providing prioritized pharmacies store suitable drug products for each one of the received purposes to the first user, wherein the prioritized pharmacies stores suitable drug products for each one of the received purposes are transmitted from the second computer; receiving the first user's command for selecting one of the prioritized pharmacies and corresponding suitable drug products for each one of the received purposes; and sending the selected pharmacy and corresponding suitable drug products for each one of the received purposes to the second computer.

According to an embodiment of the present invention, providing a method comprising: determining suitable drug products for each one of purposes received from a first computer according to a recommendation policy; determining prioritized pharmacies store the suitable drug products for each one of the purposes according to the recommendation policy; transmitting the prioritized pharmacies store the suitable drug products for each one of the purposes to the first computer; receiving the selected pharmacy and corresponding suitable drug products for each one of the received purposes from the first computer; and producing an e-prescription according to the selected pharmacy and corresponding suitable drug products for each one of the purposes.

Preferably, in order to let patient to go to just one pharmacy to take all the drug products in the e-prescription and to lower cost, the recommendation policy considers availabilities and prices of the suitable drug products for each one of the purposes provided in the prioritized pharmacies.

Preferably, in order to let patient to go to just one nearest pharmacy to take all the drug products in the e-prescription, the recommendation policy further considers a minimum distance between the prioritized pharmacies and a location of a third computer of the system configured for displaying the e-prescription.

Preferably, in order to let patient to have the e-prescription on his/her own client computer, the method further comprises sending the e-prescription to a third computer of the system configured for displaying the e-prescription.

While the invention has been described in terms of what is presently considered to be the most practical and preferred embodiments, it is to be understood that the invention needs not to be limited to the above embodiments. On the contrary, it is intended to cover various modifications and similar arrangements included within the spirit and scope of the appended claims which are to be accorded with the broadest interpretation so as to encompass all such modifications and similar structures.

What is claimed is:

1. A system, comprising:
a first computer configured for:
receiving one or more purposes for medical treatment from a first user of the first computer and transmitting the one or more purpose to a second computer;
providing prioritized suitable drug products for each one of the received purposes to the first user, wherein the suitable drug products are transmitted from the second computer;
receiving the first user's command for selecting suitable drug products for each one of the received purposes; and
sending the selected suitable drug products to the second computer; and
the second computer configured for:
determining the prioritized suitable drug products for each one of the received purposes received from the first computer according to a recommendation policy;
transmitting the prioritized suitable drug products for each one of the received purposes to the first computer;
receiving the selected suitable drug products from the first computer; and
producing an e-prescription according to the selected suitable drug products.

2. The system as claimed in claim 1, wherein the recommendation policy considers prices of the suitable drug products for each one of the purposes.

3. The system as claimed in claim 1, wherein the second computer is further configured for:
determining wherein the selected suitable drug products in the e-prescription are all available in a first pharmacy; and
sending the e-prescription to an information system of the first pharmacy in case the selected suitable drug products in the e-prescription are determined all available in the first pharmacy.

4. The system as claimed in claim 3, wherein the second computer is further configured for:
storing the e-prescription in case the selected suitable drug products in the e-prescription are not all available in the first pharmacy; and
sending the e-prescription to a third computer of the system configured for displaying the e-prescription.

5. The system as claimed in claim 3, wherein the second computer is further configured for selecting and transmitting a second pharmacy among a plurality of pharmacies where store all of the selected suitable drug products in the e-prescription to the third computer or the first computer.

6. The system as claimed in claim 5, wherein the second pharmacy is selected among the plurality of pharmacies based on a minimum distance between a location of the third computer and each of the plurality of pharmacies.

7. A method, comprising:
receiving one or more purposes for medical treatment from a first user of a first computer and transmitting the one or more purposes to a second computer;
providing prioritized suitable drug products for each one of the received purposes to the first user, wherein the suitable drug products are transmitted from the second computer;
receiving the first user's command for selecting suitable drug products for each one of the received purposes; and
sending the selected suitable drug products to the second computer.

8. The method as claimed in claim 7, wherein the recommendation policy considers prices of the suitable drug products for each one of the purposes.

9. The method as claimed in claim 7, further comprising:
determining prioritized suitable drug products for each one of purposes received from a first computer according to a recommendation policy;
transmitting the prioritized suitable drug products for each one of the received purposes to the first computer; and
receiving the selected suitable drug products from the first computer; and
producing an e-prescription according to the selected suitable drug products.

10. The method as claimed in claim 9, wherein the recommendation policy considers prices of the suitable drug products for each one of the purposes.

11. The method as claimed in claim 9, further comprises:
determining wherein the selected suitable drug products in the e-prescription are all available in a first pharmacy; and
sending the e-prescription to an information system of the first pharmacy in case the selected suitable drug products in the e-prescription are determined all available in the first pharmacy.

12. The method as claimed in claim 11, further comprises:
storing the e-prescription in case the selected suitable drug products in the e-prescription are not all available in the first pharmacy; and
sending the e-prescription to a third computer of the system configured for displaying the e-prescription.

13. The method as claimed in claim 11, further comprises:
recommending and transmitting a second pharmacy among a plurality of pharmacies where store all of the selected suitable drug products in the e-prescription to the third computer or the first computer.

14. The method as claimed in claim 13, wherein the second pharmacy is selected among the plurality of pharmacies based on a minimum distance between a location of the third computer and each of the plurality of pharmacies.

15. A system, comprising:
a first computer configured for:
receiving one or more purposes for medical treatment from a first user of the first computer and transmitting the one or more purpose to a second computer;
providing prioritized pharmacies store suitable drug products for each one of the received purposes to the first user, wherein the prioritized pharmacies stores suitable drug products for each one of the received purposes are transmitted from the second computer;
receiving the first user's command for selecting one of the prioritized pharmacies and corresponding suitable drug products for each one of the received purposes; and
sending the selected pharmacy and corresponding suitable drug products for each one of the received purposes to the second computer; and
the second computer configured for:
determining the suitable drug products for each one of the received purposes received from the first computer according to a recommendation policy;
determining the prioritized pharmacies store the suitable drug products for each one of the received purposes according to the recommendation policy;
transmitting the prioritized pharmacies store the suitable drug products for each one of the received purposes to the first computer;
receiving the selected pharmacy and corresponding suitable drug products for each one of the received purposes from the first computer; and
producing an e-prescription according to the selected pharmacy and corresponding suitable drug products for each one of the received purposes.

16. The system as claimed in claim 15, wherein the recommendation policy considers availabilities and prices of the suitable drug products for each one of the purposes provided in the prioritized pharmacies.

17. The system as claimed in claim 16, wherein the recommendation policy further considers a minimum distance between the prioritized pharmacies and a location of a third computer of the system configured for displaying the e-prescription.

18. The system as claimed in claim 15, wherein the second computer is further configured for sending the e-prescription to a third computer of the system configured for displaying the e-prescription.
